(19) United States
(12) Patent Application Publication
(10) Pub. No.: US 2021/0204991 A1
(43) Pub. Date: Jul. 8, 2021

(54) One Step Surgical Screw Fixation Technique and Design

(71) Applicants: Joshua Epstein, Hallandale Beach, FL (US); Derrick Roland, Shepherdsville, KY (US)

(72) Inventors: Joshua Epstein, Hallandale Beach, FL (US); Derrick Roland, Shepherdsville, KY (US)

(21) Appl. No.: 17/144,240

(22) Filed: Jan. 8, 2021

Related U.S. Application Data
(60) Provisional application No. 62/958,581, filed on Jan. 8, 2020.

(51) Int. Cl. A61B 17/86; A61B 17/88

(52) U.S. Cl. CPC A61B 17/863; A61B 17/8615; A61B 2017/681; A61B 17/8888; A61B 17/8897; A61B 17/864

(57) ABSTRACT

A surgical screw that is self-countersinking, self-tapping/fluted, self-drilling, and having a knurled shank designed for one step insertion. The screw has a stepped driving portion.

### CROSS REFERENCE TO RELATED APPLICATIONS

This application claims the benefit of U.S. Provisional Application No. 62/958,581, filed Jan. 8, 2020.

### FIELD OF THE INVENTION

The present Invention relates to surgical screws and more specifically to a self tapping surgical screw and a method of installing the same.

### BACKGROUND OF THE INVENTION

Many fracture and reconstruction surgeries involves open reduction and internal fixation (ORIF). An incision is made over the area to see the fractured bones. Like a jigsaw puzzle, the pieces of the broken bones are placed back together (open reduction). The broken bones are then held together (internal fixation) in this correct position with metal plates and/or screws. This internal fixation provides stability so movement can begin shortly after surgery as the fracture heals.

Plates and screws used to fix a fracture are not removed as long as they are not causing problems. Most people do not have problems with the plate and screws. In rare cases, the plate and screws can cause some pain or irritation. When this happens, the hardware may be removed after the fracture is healed.

One of the current tenets of orthopedic fixation is that bone heals better if the fracture fragments are pressed firmly together. Many orthopedic devices are designed to do just that, as well as their primary function of stabilizing the fracture in anatomic alignment. Fracture compression increases the contact area across the fracture and increases stability of the fracture. It also decreases the fracture gap and decreases stress on the orthopedic implant. This compression can be static, where the compression is produced by the fixation device alone, or dynamic, where body weight or muscle forces are used to produce additional compression.

Screws are one of the most ubiquitous hardware devices. They are used by themselves to provide fixation or in conjunction with other devices. Any screw that is used to achieve interfragmental compression is termed a lag screw. Such screws do not protect fractures from bending, rotation or axial loading forces, and other devices should are generally used to provide these functions.

The two most common types of screws are cortical and cancellous screws, as shown in FIGS. 1A and 1B. Cortical screws tend to have fine threads all along their shaft, and are designed to anchor in cortical bone. Cancellous screws tend to have coarser threads, and usually have a smooth, unthreaded portion, which allows it to act as a lag screw. These coarser threads are designed to anchor in the softer medullary bone.

Another commonly used screw is the cannulated screw, so called because of its hollow shaft shown in FIG. 1C. Although these screws have somewhat diminished pullout strength compared to conventional screws, cannulated screws have many advantages over other screws, especially the precision with which they can be placed. To place these screws, a Kirschner wire (K wire) is inserted in the area of interest. These K wires can be placed and replaced with minimal trauma to the bone until they are in optimal position. The cannulated screw is then placed over the wire and slid down to the bone surface. A special driving tool then allows the screw to be driven into the bone along the shaft of the K wire. The K wire is then withdrawn. One major complication of these screws is perforation of the articular surface when these screws are placed into a bone with their tips close to the subchondral bone. If concern about this possibility during surgery is present, contrast material may be injected through the hollow center of the screw in question—spillage into the joint cavity under fluoroscopy will be unequivocal evidence of perforation.

Other screw types include Herbert screws, Acutrak screws, and the like.

Other hardware used includes washers, plates, pins, wires, and the like.

### SUMMARY OF THE INVENTION

What is needed is a self-countersinking, self-tapping/fluted, self-drilling, and knurled shank cannulated cortical screw designed for one step insertion. Such a screw would be used for compression across an osteotomy or fracture site. Additionally disclosed is an instructional and information guide for the insertion of such a screw.

Adequate fixation is an integral piece of the surgical puzzle in both elective and non-elective procedures. This area has evolved through time with medical specialties desiring stable fixation in an efficient and reproducible manner. In foot and ankle surgery, stable osteotomies such as the chevron were once left devoid of any fixation, but in contrast, today there are a plurality of different options available including wires, staples, and even plates. However, one tried and true form of fixation remains the gold standard, the screw. Over time the screw has evolved from its original form in order to adapt to modern surgical demands. The paramount surgeons place on ease of use and efficiency, as well as the renewed focus on cost effectiveness, has led to the pervasiveness of different types of screws we see today. The constant retooling of this simple device has sharpened it into one of the most utilized, versatile, and dependable surgical constructs. However, the screw has yet to reach its full potential. A screw can become a single introducer, accompanying with it self-drilling, self-tapping and now self-countersinking abilities. No drills, no chronically dull countersinks, just one sterile packed screw.

Typically, screw diameters vary. Screw diameters are available in increments from 2 mm to 605 mm. Typical diameters include 2 mm, 2.5 mm, 3 mm, 4 mm, 4.5 mm, 6 mm, and 6.5 mm although other diameters are foreseeable. Screw lengths vary from 10 mm to 70 mm although other lengths are foreseeable.

It should be noted that the features of the disclosed cannulated screw can be applied to cortical screw, cancellous screws, Herbert screws, and Acutrak screws. Some features can also be applied to pins.

According to one aspect of the invention, the screw is sterilized.

According to one aspect of the invention, the screws are individually prepackaged in various screw sizes, which allows for more cost effective utilization of screws. Additionally, large surgical caddies and overhead costs are minimized. Alternatively, kits of multiple screws are provided.

According to one aspect of the invention a separate kit including a 0.45 Kirschner guide wire, a measuring device, and driver is provided. Such a kit can include one or more screws.

### DESCRIPTION OF THE FIGURES

FIG. 1A is a prior art cortical screw;

FIG. 1B is a prior art cancellous screw;

FIG. 1C is a prior art cannulated screw;

FIG. 2 is a screw;

FIG. 3 is a top view of the screw of FIG. 2;

FIG. 4 is a cut-away view of a screw head;

FIG. 5 depicts placing a guide wire;

FIG. 6 depicts measuring for the screw;

FIG. 7 depicts placing the screw; and

FIG. 8 depicts the installed screw.

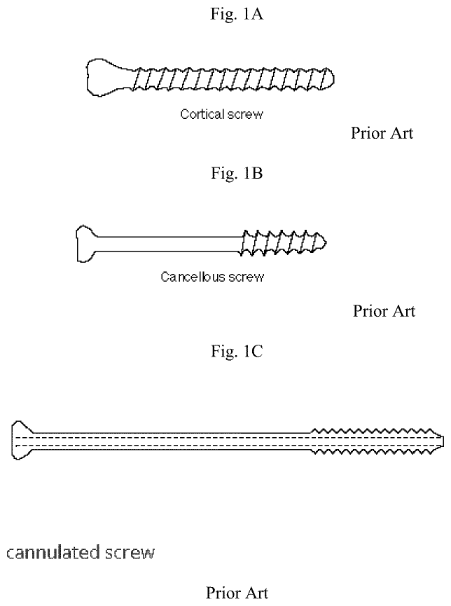

### DETAILED DESCRIPTION

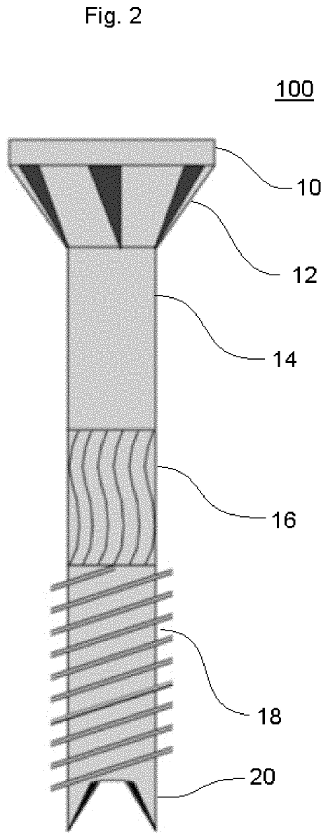

FIG. 2 is a side view of a screw 100. The screw 100 has a head 10. The head 10 is a self countersinking. The head 10 includes countersinking nibs 12. The self-countersinking head reduces risks of stress risers and over aggressive countersinking of cortical bone while limiting cortical stress. The shank 14 of the screw 100 includes a knurled portion 16. The knurled portion 16 allows for reduced friction and drag upon insertion of the screw 100.

Like all surgical screws, the screw 100 has a threaded portion 18. The threaded portion 18 is preferably a self tapping thread. The end of the screw 100 has sharp cutting flutes 20. The cutting flutes 20 allow for the self drilling and self tapping of the screw 100. Because the screw 100 is self drilling and self tapping, it has increased pull out strength so that it can provide greater overall compression than a screw that is inserted in a predrilled hole.

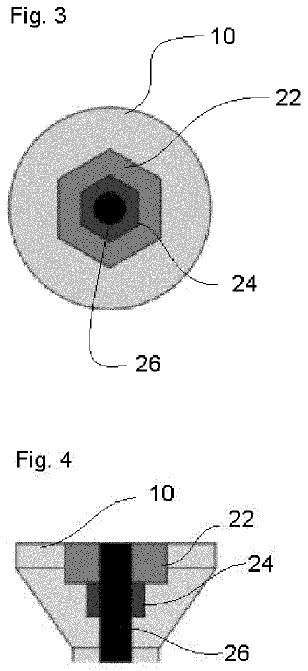

FIG. 3 is a top view the head 10 of the screw 100. As shown, the head 10 has two driving indentations 22, 24 for driving the screw 100. As shown in FIG. 4, the two driving indentations 22, 24 have different depths in the head 10. The two driving indentations 22, 24 provide additional driving torque compared to a single indent. The double drive also prevents stripping during insertion. While shown as hexagons, the driving indentations 22, 24 can be other shaped including star, marketed under the Torx name, square, double-square, triple-square, double hex, pentalobe, aster, clutch, pentagon, bristol, oval, tri-lobe, and the like. It should be noted that the two indents can be the same or different. A corresponding tool is used to drive the screw.

As seen in FIGS. 3 and 4, the screw is a cannulated screw. A thru hole 26 extends the length of the screw for use with a K wire. While shown as a cannulated screw, the in FIGS. 3 and 4, the self tapping and driving elements are applicable to any surgical screw.

Each section of the screw varies by application and overall length and diameter of the screw.

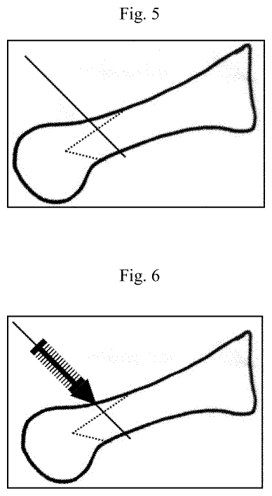

FIG. 5 depicts placing a guide wire. In use, a sterile pre-packaged instrument pack is opened. According to one aspect of the invention, the kit includes a 0.45 Kirschner guide wire, a measuring device, and driver. After identifying the optimum positioning of the screw, the supplied 0.45 Kirschner guide wire is inserted using a wire driver across the osteotomy or fracture site in the desired final position of the screw. The wire is advanced to the desired end length of the screw and is utilized to provisionally secure the capital fragment of bone. Correct guide wire placement can be confirmed utilizing intraoperative fluoroscopy.

FIG. 6 depicts measuring for the screw. Following insertion and positioning of the 0.45 guide wire, the provided measuring device is inserted over the 0.45 guide wire and the desired screw length is observed with the device flush against the near cortex of the bone. Bicortical purchase can be confirmed utilizing fluoroscopy.

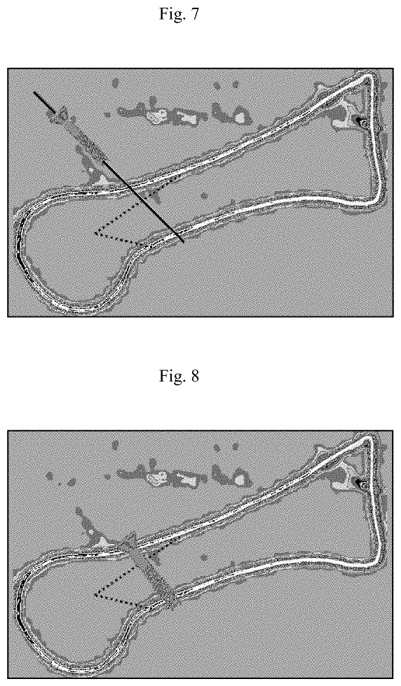

FIG. 7 depicts placing the screw. Once the desired length is measured, the sterile pre-packaged screw of the corresponding length is opened and the screw is inserted over the previously placed guide wire. The screw is then advanced on the guide wire utilizing the provided driver until the near cortex is engaged by the threads of the screw. The screw is advanced by turning the driver in a clockwise motion. Prior to final tightening of the screw and achievement of compression, the self-countersinking heads will begin to engage the near cortex and will cut a recessed area of cortical bone so that the screw head sits flush with the near cortex. The screw should be advanced until the screw threads completely cross the osteotomy or fracture site and 1-2 screw threads cross the far cortex for increased pull-out strength. At this point the self-countersinking head will be securely seated in the near cortex of bone, and the osteotomy or fracture will be compressed. The process of under drilling, over drilling, tapping, and countersinking is performed in one step and the screw is inserted in one motion.

FIG. 8 depicts the installed screw, not to scale or fully seated. Prior to final tightening of the screw and achievement of compression, the self-countersinking heads will engage the near cortex and will cut a recessed area of cortical bone so that the screw head sits flush with the near cortex. The screw should be advanced until the screw threads completely cross the osteotomy or fracture site and 1-2 screw threads cross the far cortex for increased pull-out strength

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve substantially the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A screw comprising:
a head having countersinking nibs;
a shank that extends from the head and includes:
a knurled portion; and
a threaded portion; and
cutting flutes opposite the head.

2. The screw of claim 1, wherein the head has a stepped driving portion.

3. The screw of claim 2, wherein the stepped driving portion comprises a Torx, square, double-square, triple-square, double hex, pentalobe, aster, clutch, pentagon, bristol, oval, and tri-lobe configuration.

4. The screw of claim 3, wherein the stepped driving portion are a same or different.

5. The screw of claim 1, wherein the screw is a one of a cannulated screw and a self tapping screw.

6. The screw of claim 1, further defining a thru hole that extends a length of the screw and configured for a K wire.

7. A kit comprising:
a screw comprising:
a head having countersinking nibs;
a shank that extends from the head and includes:
a knurled portion; and
a threaded portion; and
cutting flutes opposite the head,
a measuring device; and
a driver for the screw.

8. The kit of claim 7, further comprising a Kirschner guide wire.

9. The kit of claim 8, wherein the Kirschner guide wire is a 0.45 Kirschner guide wire.

10. The kit of claim 8, wherein the kit is a sterile pre-packaged instrument pack

11. The kit of claim 7, wherein the driver for the screw comprises a stepped driving portion configured to drive one or more of a Torx, square, double-square, triple-square, double hex, pentalobe, aster, clutch, pentagon, bristol, oval, and tri-lobe configuration.

12. The screw of claim 11, wherein the stepped driving portion are a same or different
